(19) United States
(12) Patent Application Publication
(10) Pub. No.: US 2023/0402184 A1
(43) Pub. Date: Dec. 14, 2023

(54) Monitoring Device

(71) Applicant: Sanofi, Paris (FR)

(72) Inventors: Michael Helmer, Frankfurt am Main (DE); Martin Vitt, Frankfurt am Main (DE)

(21) Appl. No.: 18/035,664

(22) PCT Filed: Nov. 16, 2021
(86) PCT No.: PCT/EP2021/081765

§ 371 Date: May 5, 2023

(30) Foreign Application Priority Data
Nov. 17, 2020 (EP) 20315453.9

(51) Int. Cl. G16H 50/30; G16H 20/17

(52) U.S. Cl. CPC G16H 50/30; G16H 20/17

(57) ABSTRACT

A mobile device including a memory and a processing arrangement is described. The memory stores instructions which, when executed by the processing arrangement, cause the device to receive dose information including a previous dose ejected by an injection device; determine a measure of wellbeing for a patient based on received wellbeing data; determine at least one of a measure of fitness for the patient based on physiological data received from fitness tracking device and a measure of bodyweight for the patient based on weight data received from a balance; determine an adjusted medicament dose based at least in part on the dose information, the measure of wellbeing, and the at least one of the measure of fitness and the measure of bodyweight; and output the adjusted medicament dose.

### CROSS REFERENCE TO RELATED APPLICATIONS

The present application is the national stage entry of International Patent Application No. PCT/EP2021/081765, filed on Nov. 16, 2021, and claims priority to Application No. EP 20315453.9, filed on Nov. 17, 2020, the disclosures of which are incorporated herein by reference.

### TECHNICAL FIELD

The present disclosure relates to monitoring a patient during drug treatment. In particular, the disclosure relates to a device and system for monitoring a patient during drug treatment, and a method for operating the device.

### BACKGROUND

A variety of diseases exist which require regular treatment, for instance, by injection of a medicament. Such injections can be performed by using injection devices, which are applied either by a healthcare professional (HCP) or by patients themselves. As an example, type-1 and type-2 diabetes can be treated by patients themselves by injection of insulin doses, for example once or several times per day. Similarly, at a preliminary stage before diabetes, patients who are overweight or obese can administer by injection treatment for chronic weight management.

Unfortunately, in the course of drug treatment using some drugs the patient may experience side-effects, which may result either from the drugs themselves or the condition for which they are being treated. These side-effects could include, for instance, weight changes, greater susceptibility to contracting other diseases or an overall deterioration in general health conditions.

Accordingly, where patients require regular or long-term treatment, there is a need to provide a system that can support the treatment and monitor the patient's overall well-being in response to that treatment. In particular, there is a need to determine any changes in the patient's health in response to a treatment regimen and to encourage good patient habits in adhering to the treatment regimen. This is important as changes in the patient's response to the treatment may require, for instance, the nature of the treatment, such as the dosing regimen, to be adjusted or additional treatment to be introduced.

A significant proportion of patients with type-2 diabetes are overweight or obese. Many of the available classes of treatment options are actually associated with promoting weight gain, which can be a distressing side effect for patients already struggling with excess bodyweight and one which may adversely impact adherence to therapy. SAR425899 is a dual agonist of the glucagon-like peptide-1 and glucagon receptors (GLP-1/GCR agonist), and is being developed for simultaneous diabetes management and chronic weight management for those who are overweight or obese. Glucagon-like peptide enhances patient satiety with a subsequent reduction in food intake, thereby promoting weight loss. A regimen of SAR425899 injections can therefore be used to control glucagon levels of a patient and assist with weight management.

Aspects of the present disclosure have been conceived with the foregoing in mind.

### SUMMARY

According to a first aspect of the present disclosure, there is provided a mobile device comprising a memory and a processing arrangement, the memory storing instructions which, when executed by the processing arrangement, cause the device to:
receive dose information comprising a previous dose ejected by an injection device;
determine a measure of wellbeing for a patient based on received wellbeing data;
determine at least one of:
   a measure of fitness for the patient based on physiological data received from fitness tracking device; and
   a measure of bodyweight for the patient based on weight data received from a balance;
determine an adjusted medicament dose based at least in part on the dose information, the measure of wellbeing, and the at least one of the measure of fitness and the measure of bodyweight; and
output the adjusted medicament dose.

The mobile device may be for performing a titration.

Determining the adjusted medicament dose may comprise adjusting the previous dose comprised in the received dose information. Determining the adjusted medicament dose may comprise adjusting the previous dose based on the measure of wellbeing and the at least one of the measure of fitness and the measure of bodyweight. Determining the adjusted medicament dose may comprise increasing, decreasing or maintaining the previous dose based on the measure of wellbeing and increasing, decreasing or maintaining the previous dose based on the at least one of the measure of fitness and the measure of bodyweight,

The instructions, when executed by the processing arrangement, may further cause the device to output at least the wellbeing data and the dose information comprising the previous dose to a server arrangement for storage.

The physiological data may comprise pulse data and the instructions, when executed by the processing arrangement, may cause the device to determine the measure of fitness based on the pulse data.

The physiological data may comprise blood pressure data and the instructions, when executed by the processing arrangement, may cause the device to determine the measure of fitness based on the blood pressure data.

The physiological data may comprise step count data and the instructions, when executed by the processing arrangement, may cause the device to determine the measure of fitness based on the step count data.

The instructions, when executed by the processing arrangement, may further cause the device to output at least one of the measure of wellbeing, the measure of fitness and the measure of bodyweight for display.

The at least one of the measure of wellbeing, the measure of fitness and the measure of bodyweight output for display may be displayed along with one or more previous measures of wellbeing, measures of fitness or measures of bodyweight.

The instructions, when executed by the processing arrangement, may further cause the device to: receive side effect data indicative of one or more side effects experienced by the patient; and determine the adjusted medicament dose based at least in part on the side effect data.

The instructions, when executed by the processing arrangement, may further cause the device to: determine whether the measure of fitness meets a first predetermined threshold; determine whether the measure of bodyweight meets a second predetermined threshold; and in response to determining that the measure of fitness meets the first predetermined threshold or that the measure of bodyweight meets the second predetermined threshold, output a virtual award for display at the mobile device.

The instructions, when executed by the processing arrangement, may further cause the device to receive food intake information input by a user and store the received food intake information.

The instructions, when executed by the processing arrangement, may further cause the device to publish data corresponding to at least one of the measure of wellbeing, the measure of fitness and the measure of bodyweight to a social media platform.

According to a second aspect of the present disclosure, there is provided a system comprising a mobile device according to the first aspect, the system further comprising:
a fitness tracking device configured to transmit physiological data to the mobile device; and
a balance configured to transmit weight data to the mobile device,
wherein the mobile device is configured to determine an adjusted medicament dose based at least in part on the physiological data and the weight data received by the mobile device.

The system may further comprise an injection device configured to transmit dose information to the mobile device, wherein the device is configured to determine the adjusted medicament dose based at least in part on the dose information.

According to a third aspect of the present disclosure, there is provided a computer implemented method comprising:
receiving dose information comprising a previous dose ejected by an injection device;
determining a measure of wellbeing for a patient based on received wellbeing data;
determining at least one of:
   a measure of fitness for the patient based on physiological data received from fitness tracking device; and
   a measure of bodyweight for the patient based on weight data received from a balance;
determining an adjusted medicament dose based at least in part on the dose information, the measure of wellbeing, and the at least one of the measure of fitness and the measure of bodyweight; and
outputting the adjusted medicament dose.

The method may be a method for performing a titration.

According to a fourth aspect of the present disclosure, there is provided a computer-readable storage medium comprising instructions which, when executed by a computer, cause the computer to carry out the method of the third aspect.

Embodiments of the disclosure will now be described, by way of example only, with reference to the accompanying drawings.

### BRIEF DESCRIPTION OF THE FIGURES

In the Figures:

FIG. 1 is a schematic showing components of a mobile device according to embodiments of the present disclosure;

FIG. 2 is a schematic showing a system comprising the mobile device of FIG. 1, according to embodiments of the present disclosure;

FIG. 3 is a schematic showing data flows in the system of FIG. 2;

FIG. 4 is a schematic showing data output as a plurality of graphs on the display of the device of FIG. 3;

FIG. 5 is a flowchart illustrating a method of titration according to embodiments of the present disclosure;

FIG. 6 is a flowchart illustrating a method of drug treatment monitoring according to embodiments of the present disclosure.

### DETAILED DESCRIPTION

In the following disclosure a computer program will be described having machine readable instructions that when executed by a processing arrangement causes the processing arrangement to initiate various operations. In the following exemplary embodiments the computer program is implemented in a mobile device and the computer program is in the form of an application. According to the following exemplary embodiments, the mobile device is a mobile phone, such as a smartphone. Advantageously, the monitoring application may be a distinct application. The monitoring application may be provided in the mobile device on manufacture or may be downloaded into the mobile device by a user, for instance from an application market place or application store. The mobile device may, however, take a different form such as a tablet, laptop, smartglasses or the like.

In brief, the computer program as implemented in the mobile device provides a means for monitoring a patient during drug treatment. The drug may be SAR425899 and the treatment may be for chronic weight management, however the drug and the treatment are not limited to such examples, and aspects of the present disclosure may instead be applied to other drugs and/or treatments.

The monitoring application could be provided to support a variety of clinical studies. These could include, for instance, clinical studies related to diabetes, chronic weight control, cardio vascular conditions, rheumatism or psoriasis. The terms user and patient may be used interchangeably throughout this disclosure.

Aspects of the present disclosure provide support for patient treatment. Aspects of the present disclosure may encourage patient adherence to a drug treatment regimen, leading to more effective, faster and, in some cases, safer treatment outcomes. In the example of a treatment regimen involving the use of SAR425899 or the like, aspects of the present disclosure may support further weight reduction of a patient. An improvement in patient adherence may be brought about by improvements in the titration of dose titration associated with the present disclosure. Aspects of the present disclosure may help encourage patient adherence by allowing the patient to visualize their own medical statistics and monitor data associated with their treatment, such as fitness levels and body mass index (BMI), over time. Patients may also be encouraged through sharing of their statistics to social media platforms for motivational and/or social feedback and encouragement.

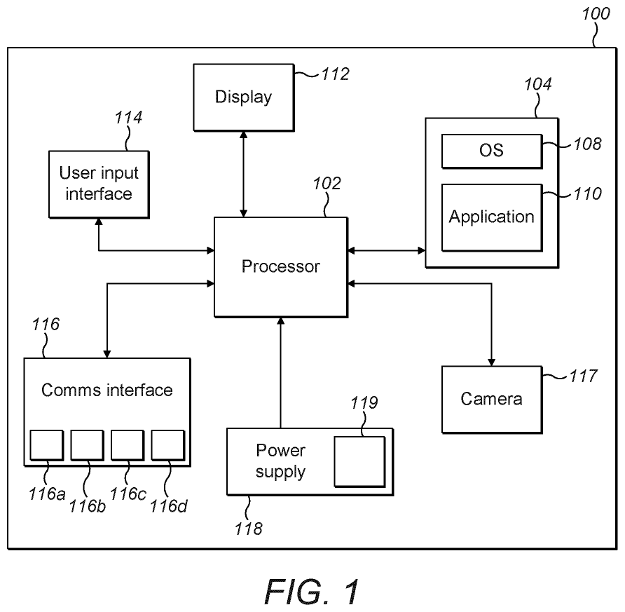

FIG. 1 is a schematic of some of components of the mobile device 100 on which the computer program can operate. The mobile device 100 includes a processing arrangement 102. The processing arrangement 102 may comprise one or more processors, logic circuits or the like. The processing arrangement 102 controls operation of the other hardware components of the mobile device 100. The processing arrangement 102 and other hardware components may be connected via a system bus (not shown). Each hardware component may be connected to the system bus either directly or via an interface. The various method steps disclosed throughout this application may be performed, or caused to be performed, by the processing arrangement 102, unless where stated otherwise.

The mobile device 100 comprises a display 112, which may be any suitable type of display such as a liquid crystal display (LCD), thin film transistor (TFT) display, organic light emitting diode (OLED) display, ePaper, or the like. The processing arrangement 102 is configured to provide instructions to the display 112 that cause the display 112 to provide visual information to a user. In some cases the processing arrangement 102 provides the instructions to the display 112 via a display driver (not shown).

The device 100 further comprises a user input interface 114. The user input interface 114 is a means through which a user may provide an input to the mobile device 100, and in particular the processing arrangement 102. In some instances, the user input interface 114 may comprise a tactile user interface for receiving tactile inputs from the user. For example, the tactile user interface may comprise one or more buttons or a touch sensitive panel. The touch sensitive panel may be a resistive touch sensitive panel or a capacitive touch sensitive panel of any kind, for example. In some cases, the display 112 may be a touch sensitive display that also comprises the user input interface 114. This may be the case where the device 100 is a smartphone. In some examples, the user input interface 114 may comprise an audio user interface which is configured to receive voice commands as input from the user, for example via a microphone. In this way, the user may provide input into user input interface 114 via voice commands. The voice commands may be parsed and converted by the processing arrangement 102 or a different processing component of the device 100 into machine language instructions.

The mobile device 100 also includes a communications interface 116 for transmitting data to other electronic devices or systems and receiving data from other electronic device or systems, as described later. The communications interface 116 may take any suitable form for providing wireless and/or wired communication. For example, the communications interface 116 may comprise at least one of a Bluetooth interface 116a, a WiFi interface 116b, an Near Field Communication (NFC) interface 116c, and a wired interface 116d such as a USB (e.g. USB-C) interface, although other suitable types of wired or wireless communications interface 116 may be used.

The mobile device 100 may optionally comprise a camera arrangement 117 comprising one or more cameras. Any suitable camera may be employed. The processing arrangement 102 may be configured to process one or more images captured by the camera arrangement 117, for example to extract data from the one or more images.

The mobile device 100 also comprises a power supply 118 for supplying power to the various electronic components of the mobile device 100. The power supply 118 may comprise a battery 119, or it may receive power through another means such as wireless power transfer or a power cable.

The processing arrangement 102 is configured to send and receive signals to and from the other components of the device 100 in order to control operation of the other components. For example, the processing arrangement 102 controls the display of content on the display 112 and receives signals as a result of user inputs via the user input interface 114.

The mobile device 100 comprises a memory 104, for example a working or volatile memory such as Random Access Memory (RAM), and a non-volatile memory. The processing arrangement 102 may access RAM in order to process data and may control the storage of data in memory 104. The RAM may be a RAM of any type, for example Static RAM (SRAM), Dynamic RAM (DRAM) or a Flash memory. The non-volatile memory stores an operating system 108 and the application 110, as well as storing data files and associated metadata. The memory 104 may comprise a non-volatile memory of any kind such as a Read Only Memory (ROM), a flash memory and a magnetic drive memory.

The processing arrangement 102 operates under control of the operating system 108. The operating system 108 may comprise code relating to hardware such as the display 112 and the communications interface 116, as well as the basic operation of the mobile device 100. The operating system 108 may also cause activation of other software modules stored in the memory 104, in addition to or instead of the application 110.

Other standard or optional components of the mobile device 100 such as transceivers and audio transducers are omitted for brevity, but may be present.

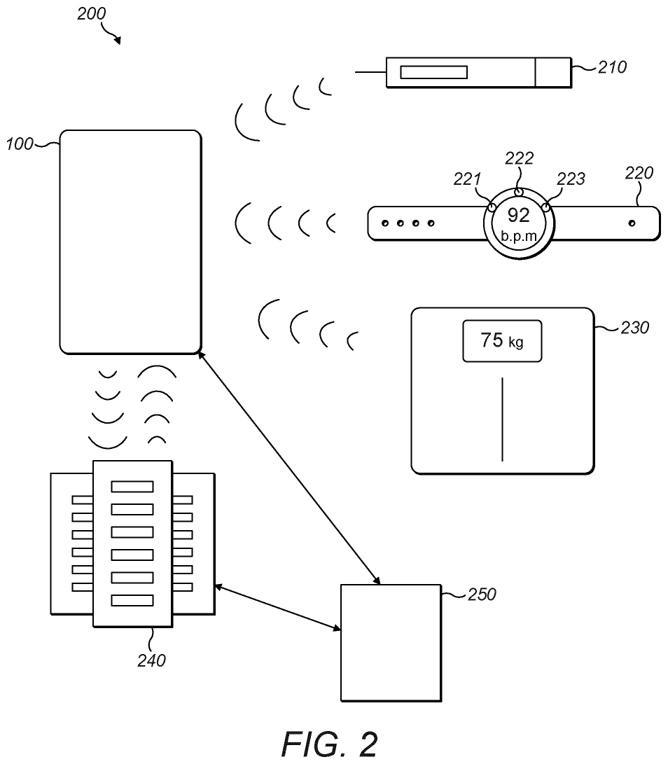

FIG. 2 shows the mobile device 100 as part of a monitoring system 200. The system 200 can be used by a patient to monitor their treatment regimen. As should become clear throughout this disclosure, the monitoring system 200 can be used to collect and store data associated with the patient and the treatment regimen such as fitness related data, bodyweight data, dose information, patient wellbeing and side effect data. The data may be presented to the user so that they may track their progress during the treatment regimen and be encouraged to adhere to the regimen. The data may be used to provide improved titration of a drug dose for the patient, which may improve treatment outcomes, encourage patient adherence, and empower the patient to perform their own titration rather than entirely relying on a health care professional.

The system 200 comprises an injection device 210, a fitness tracking device 220, a balance 230 and a server arrangement 240. The mobile device 200 is able to communicate with one or more of the injection device 210, fitness tracking device 220, electronic balance 230 and server arrangement 240 as discussed below, sending and/or receiving data via the communications interface 116.

The injection device 210 may be any suitable form of device for administering a dose of medicament to a patient. For example, the injection device 210 may be an injection pen, an autoinjector, a pump patch, an infusion device, or the like. The injection device 210 is used to inject a dose of medicament (drug) into the patient/user. The injection device 210 is therefore used by the patient/user as part of their treatment regimen.

The injection device 210 has electronic circuitry (not shown) that can transmit dose information to the mobile device 100, to be received via the communications interface 116 of the mobile device 100. The dose information comprises a dose of medicament ejected from, or due to be ejected from, the injection device 210. In other words, the dose may be the dose that has actually been injected by the injection device 210 into the patient, or a dose that has been programmed into the injection device 210 ready for subsequent ejection. The dose may be expressed as a dose value, such as a number of units of medicament.

In addition to a dose, the dose information may include further information such as a time and/or date that the dose was injected, as determined by the injection device 210.

The dose information may be transmitted from the injection device 210 to the communications interface 116 of the mobile device 100 wirelessly, for example using a Bluetooth, NFC or WiFi protocol, or any other suitable wireless protocol. In other examples, the dose information may be transmitted over a wired communication link to the communications interface 116 of the mobile device 100. In some examples the injection device 210 may transmit the dose information to the mobile device 100 in response to receipt of a signal sent by the mobile device 100 to the injection device 210, as discussed later. In other examples the injection device 210 may transmit the dose information to the mobile device 100 in response to receipt of a user input at the injection device 210, such as in response to user actuation of an injection mechanism of the injection device 210 that causes dispensing of the medicament, or in response to a user pressing a button on a user interface of the injection device 210. In some examples, the injection device 210 may transmit the dose information automatically, for example according to a predetermined schedule (e.g. every day).

The dose information may be received by the mobile device 100 in manners other than via the communications interface 116. For example, the camera arrangement 117 of the mobile device 100 may be used to capture an image of the injection device 210 and the processing arrangement 102 of the mobile device 100 may extract the dose information from the image. For example, the image may contain a visible representation of the dose ejected by or dialled into the injection device 210, such as a display of the injection device 210 showing the dose on a dialling sleeve. The processing arrangement 102, or a different device, may identify the dose from the image of the dialling sleeve.

The system 200 may also comprise a fitness tracking device 220. The fitness tracking device 220 is shown in FIG. 2 as a fitness tracking smartwatch, but any suitable form of fitness tracking device 220 may be used instead. In some examples, functions of the fitness tracking device 220 may be performed by a plurality of devices such as a plurality of fitness bands, smartwatches or monitors. The fitness tracking device 220 is configured to monitor one or more physiological characteristics of a patient, in particular the patient receiving the treatment from the injection device 210. The physiological characteristics can be indicative of a fitness level of the patient. The physiological characteristics may comprise one or more of a heart rate (pulse), blood pressure or step count, although other physiological characteristics may be used.

The fitness tracking device 220 comprises one or more sensors for measuring the physiological characteristics of the patient to determine physiological data indicative of that measured characteristic. For example, the fitness tracking device 220 may comprise one or more of a heart rate sensor 221 for measuring a heart rate of the patient, a blood pressure sensor 222 for measuring a blood pressure of the patient, and/or a pedometer 223 for measuring a step count of the patient.

The fitness tracking device 220 may measure one or more of the physiological characteristics continuously. For example, the fitness tracking device may measure the patient's heart rate and/or step count continuously. The fitness tracking device 220 may measure one or more of the physiological characteristics at a predetermined discrete interval. For example, the fitness tracking device may take a measurement of the patient's blood pressure once daily. The fitness tracking device 220 may measure one or more of the physiological characteristics in response to receipt of an input such as a user input at the fitness tracking device 220 or in response to receipt of a signal transmitted from the mobile device 100. For example, the fitness tracking device may measure the patient's heart rate in response to the user pressing a button on the fitness tracking device 220.

The physiological data measured by the fitness tracking device 220 may be stored in a memory of the fitness tracking device 220 and/or sent to another electronic device for storage, such as the mobile device 100, a different electronic device, or a server. The physiological data may be processed by the fitness tracking device 220, mobile device 100, different electronic device, and/or server.

The fitness tracking device 220 may transmit the physiological data to the mobile device 100 to be received via the communications interface 116 of the mobile device 100. For example, the fitness tracking device 220 may transmit one or more of heart rate data, blood pressure data and step count data to the mobile device 100. The fitness tracking device 220 may transmit the physiological data associated with the one or more physiological characteristics wirelessly to the communications interface 116, for example using a Bluetooth, NFC or WiFi protocol, or any other suitable wireless protocol. In other examples, the physiological data may be transmitted over a wired communication link to the communications interface 116 of the mobile device 100.

In some examples the fitness tracking device 220 may transmit the physiological data to the mobile device 100 in response to receipt of a request sent by the mobile device 100 to the fitness tracking device 220, as discussed later. In other examples the fitness tracking device 220 may transmit the physiological data to the mobile device 100 in response to receipt of a user input at the fitness tracking device 220, such as a user providing an input at a user interface of the fitness tracking device 220. In some examples the physiological data may be automatically transmitted from the fitness tracking device 220 to the mobile device 100, rather than being transmitted in response to a request such as an input from the user or a request from the mobile device 100. For example, the fitness tracking device 220 may transmit physiological data indicative of one or more of the physiological characteristics every hour, every day or every week. Physiological data associated with some of the physiological characteristics may be transmitted at a greater frequency than physiological data associated with the other physiological characteristics. For example, data indicative of a heart rate may be transmitted every minute, while blood pressure data may be transmitted daily.

In addition to the data indicative of the one or more physiological characteristics, the fitness tracking device 220 may also transmit to the mobile device 100 time and/or date information associated with the one or more physiological characteristics. For example, the information may include a time and/or date that the physiological characteristic was measured by the fitness tracking device 220.

The physiological data associated with one or more of the physiological characteristics may be received by the mobile device 100 in manners other than via the communications interface 116. For example, the camera arrangement 117 of the mobile device 100 may be used to capture an image of the fitness tracking device 220 and the processing arrangement 102 of the mobile device 100 may extract the physiological data from the image. For example, the image may contain a visible representation of the physiological data associated with one or more physiological characteristics, such a measured heart rate, blood pressure and/or step count shown on a display of the fitness tracking device 220.

The system 200 may also comprise a balance 230, also known as weighing scales, used for measuring a weight of the patient. The mobile device 100 is able to receive weight data from the balance 230, the weight data being indicative of a weight of the patient. The balance 230 may be an electronic balance 230 comprising electronic circuitry, which is able to measure a weight of the patient and transmit the weight data to the mobile device 100 wirelessly, for example using a Bluetooth, NFC or WiFi protocol, or any other suitable wireless protocol. In other examples, the weight data may be transmitted over a wired communication link to the communications interface 116 of the mobile device 100. In some examples the balance 230 may transmit the weight data to the mobile device 100 in response to receipt of a request sent by the mobile device 100 to the balance 230. In other examples the balance 230 may transmit the weight data to the mobile device 100 in response to receipt of a user input at the balance 230, such as detection of a user stepping onto the balance 230, or a user pressing a button on a user interface of the balance 230.

In addition to a weight value, the weight data may include further information such as a time and/or date that the weight was measured by the balance 230.

The weight data may be received by the mobile device 100 in manners other than via the communications interface 116. For example, the camera arrangement 117 of the mobile device 100 may be used to capture an image of the balance 230 and the processing arrangement 102 of the mobile device 100 may extract the weight data from the image. For example, the image may contain a visible representation of the weight measured by the balance 230, such as a weight of the patient shown on a display of the balance 230.

The system 200 may also comprise a server arrangement 240 comprising one or more servers. The one or more servers may be part of a distributed computing arrangement such as a cloud computing arrangement. The mobile device 100 may communicate with the server arrangement 240 via the communications interface 116 using any suitable means of wired or wireless communication, for example through the Internet, a cellular network or local area network (LAN), although other arrangements may be used. In some examples the server arrangement 240 may instead comprise a personal computing device such as another mobile device, a laptop, a desktop computer, a tablet or the like. The mobile device 100 may transmit data to the server arrangement 240 and/or receive data from the server arrangement 240, as discussed later. The mobile device 100 may transmit the data to the server arrangement 240 for storage or for processing of the data.

In some examples, one or more aspects disclosed herein as being performed by the mobile device 100 may additionally or alternatively be performed by the server arrangement 240. For example, rather than performing a processing step on the mobile device 100 itself, the mobile device 100 may instead transmit data to the server arrangement 240 for performing the processing, with the mobile device 100 subsequently receiving the result of the processing from the server arrangement 240. This may allow the mobile device 100 to use improved processing capabilities of the server arrangement 240 in order to process data faster and to reduce the processing burden on the mobile device 100. Alternatively or additionally, where it has been disclosed herein that one or more of the injection device 210, fitness tracking device 220 and balance 230 transmit data or information to the mobile device 100 or receive data or information from the mobile device 100, this may not be via direct communication with the mobile device 100 but may instead be via an intermediary such as the server arrangement 240.

In some examples, the mobile device 100 may also be able to communicate with a social media platform 250, such as a social media platform 250 provided by Facebook or Twitter. The mobile device 100 may communicate with the social media platform 250 via the server arrangement 240 or through any other suitable type of network arrangement. As discussed later, the mobile device 100 may be able to post data to the social media platform 250, which one or more other users of the social media platform 250 may then be able to view. The mobile device 100 may also be able to receive and view information sent from the social media platform 250 to the mobile device 100.

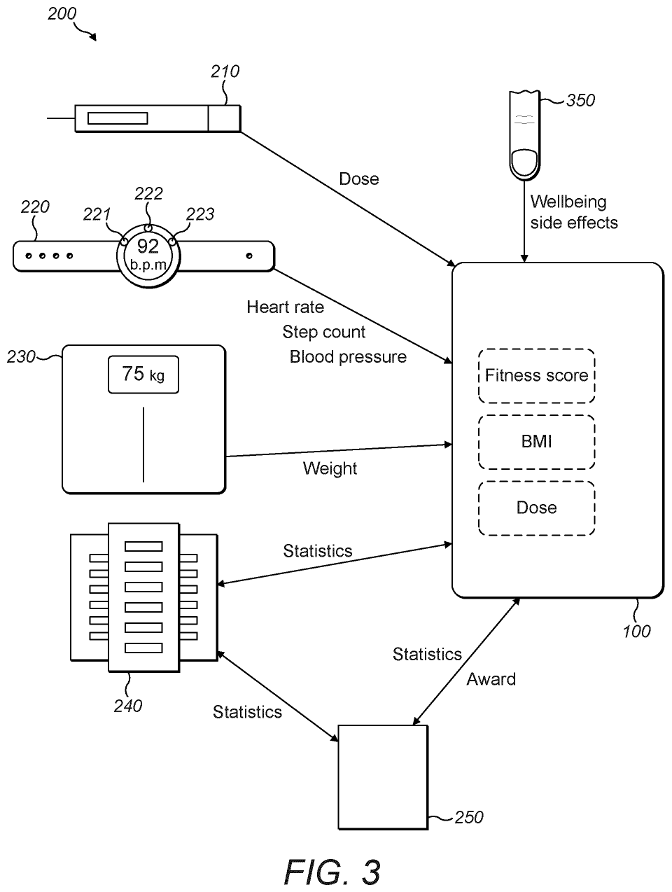

FIG. 3 is a schematic illustration of the system 200 of FIG. 2 showing the various exemplary data flows between the components of the system 200.

As illustrated in FIG. 3, the device 100 can receive dose information from the injection device 210, as discussed previously. The dose information may include a dose value and in some examples a time and/or date that the dose was injected, as measured by the injection device 210.

As seen in FIG. 3, the device 100 can receive physiological data associated with one or more physiological characteristics from the fitness tracking device 220 such as, but not limited to, a heart rate of the patient, a step count of the patient, or a blood pressure of the patient. Each time physiological data has been received, it can be stored in the memory 104 of the device 100 and/or transmitted to the server arrangement 240 for storage. The physiological data may be stored in association with a timestamp representing a time and/or date at which the physiological data was received, or a time and/or date that one or more of the measurements made by the fitness tracking device 220 were performed. The timestamp may comprise timestamp information transmitted by the fitness tracking device 220 to the mobile device 100.

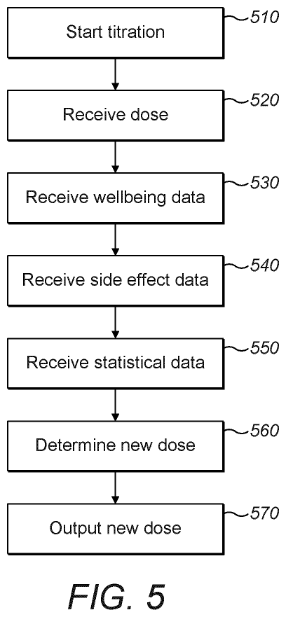

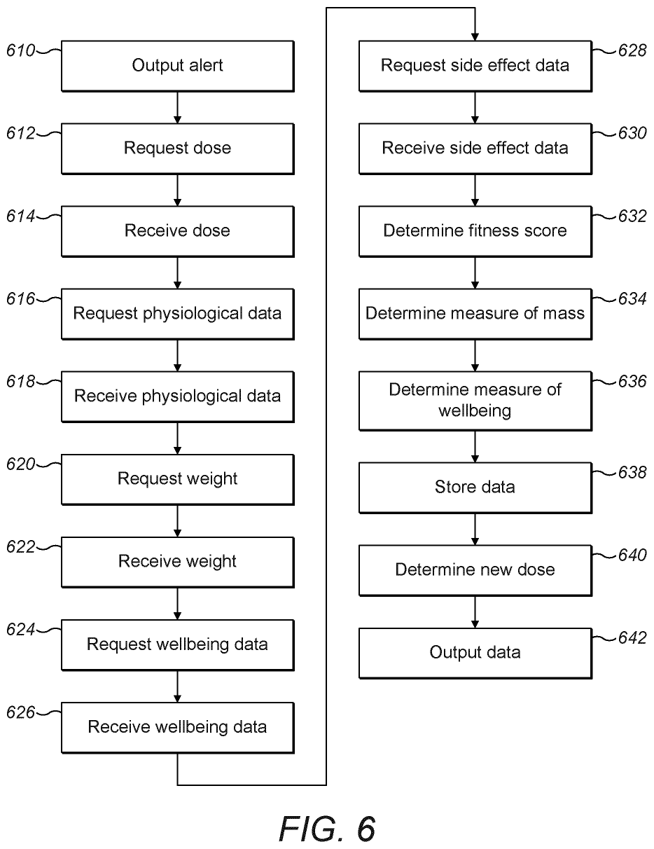

The device 100 may determine a measure of fitness for the patient based at least in part on the physiological data received from the injection device 100. The measure of fitness provides a quantitative indication of the physiological fitness of the patient and may, for example, be a fitness score. For example, the device 100 may determine a measure of fitness based at least in part on one or more of a heart rate, step count and blood pressure received from the fitness tracking device 220. The measure of fitness may be normalized. As an example, the measure of fitness may be calculated as a score between 0 and 100, where 0 is indicative of the patient having the lowest level of fitness and 100 is indicative of the patient having the highest level of fitness. A new measure of fitness may be calculated periodically. For example, a measure of fitness may be calculated according to a predetermined schedule (e.g. daily), in response to receipt of new physiological data from the fitness tracking device 220 (e.g. in response to new blood pressure data being received), in response to receipt of a user input at the user input interface 114, or as part of a method such as a method discussed in relation to FIG. 5 and FIG. 6. The measure of fitness may therefore change over time, for example increasing as the fitness of the patient increases, and decreasing if the fitness of the patient decreases.

The different types of physiological data may be weighted when determining the measure of fitness. Some types of physiological data may be more greatly weighted than other types such that they have a larger influence on the determined measure of fitness. For example, blood pressure data may be more greatly weighted than step count data such that improvements in patient blood pressure are more likely to lead to a greater improvement in the measure of fitness than improvements in step count.

The measure of fitness may be determined based on the most recent physiological data received by the mobile device 100, or it may be determined at least in part based on historical physiological data. As an example, a moving average of heart rate data may be used to determine the measure of fitness. In some cases the moving average may be based on a predetermined number of the most recently received physiological data measurements (e.g. the last ten measurements of heart rate received by the mobile device 100). In some cases the moving average may be based on an average over a predetermined time period, for example the average heart rate over the last seven days. The physiological data may be weighted such that more recent physiological data is given a greater weighting than older physiological data.

Each time a measure of fitness has been calculated, it can be stored in the memory 104 of the device 100 and/or transmitted to the server arrangement 240 for storage. The measure of fitness may be stored in association with a timestamp representing a time and/or date at which the measure of fitness was determined, or a time and/or date that one or more of the measurements made by the fitness tracking device 220 were performed. The stored measurements of fitness and the associated timestamp information may provide a record of the patient's changing physiological fitness. The record may be used to assess how the patient's physiological health has changed over time, for example whether it is improving or worsening as the treatment regime progresses. The record of measurements of fitness may be output to the user as a visualization, as discussed later, or transmitted to the server arrangement 240 for analysis.

As seen in FIG. 3, the device 100 can receive weight data from the balance 230. Each time weight data has been received, it can be stored in the memory 104 of the device 100 and/or transmitted to the server arrangement 240 for storage. The weight data may be stored in association with a timestamp representing a time and/or date at which the weight data was acquired, or a time and/or date at which the weight data was received by the mobile device 100. The timestamp may comprise timestamp information transmitted by the balance 230 to the mobile device 100.

The device 100 may determine a measure of bodyweight of the patient based at least in part on the weight data received from the balance 230. The measure of bodyweight provides a quantitative indication of the weight of the patient and may, for example, be a body mass index (BMI) value. If the device 100 determines a BMI for the patient then it may do so based on the weight data and based on a height of the patient. The height of the patient may have been previously stored in the device 100, for example in the memory 104 of the device 100, or the device 100 may prompt the user to enter a height via the user input interface 114. In some examples the height may be received by the device 100 from the server arrangement 240, from a different server, or the like.

A new measure of bodyweight may be calculated periodically. For example, a measure of bodyweight may be calculated according to a predetermined schedule (e.g. daily), in response to receipt of new weight data from the balance 230, in response to receipt of a user input at the user input interface 114, or as part of a method such as a method discussed in relation to FIG. 5 and FIG. 6. The measure of bodyweight may therefore change over time, for example decreasing during the course of treatment.

The measure of fitness may be determined based on the most recent weight data received by the mobile device 100, or it may be determined at least in part based on historical weight data. As an example, a moving average of weight data may be used to determine the measure of bodyweight. In some cases the moving average may be based on a predetermined number of the most recently received weight data measurements (e.g. the last five measurements of weight received by the mobile device 100). In some cases the moving average may be based on an average over a predetermined time period, for example the average weight over the last seven days. The historical weight data may be weighted such that more recent weight data is given a greater weighting than older weight data.

Each time a measure of bodyweight has been calculated, it can be stored in the memory 104 of the device 100 and/or transmitted to the server arrangement 240 for storage. The measure of bodyweight may be stored in association with a timestamp representing a time and/or date at which the measure of bodyweight was determined, or a time and/or date that one or more of the weight measurements were performed by the balance 230. The stored measurements of bodyweight and the associated timestamp information may provide a record of the patient's changing bodyweight. The record may be used to assess how the patient's bodyweight has changed over time, for example whether it is increasing or decreasing as the treatment regime progresses. The record of measurements of bodyweight may be output to the user as a visualization, as discussed later, or transmitted to the server arrangement 240 for analysis.

The device 100 can also receive wellbeing data and side effect data from the user 350. The user 350 enters the wellbeing data and side effect data into the device 100, for example via the user input interface 114.

The wellbeing data comprises data indicative of a current wellbeing of the patient, such as the happiness or mood of the patient. Each time wellbeing data has been received, it can be stored in the memory 104 of the device 100 and/or transmitted to the server arrangement 240 for storage. The wellbeing data may be stored in association with a timestamp representing a time and/or date at which the wellbeing data was received.

The device 100 may determine a measure of wellbeing for the patient based at least in part on the wellbeing data received from the user 350. The measure of wellbeing provides a quantitative indication of the wellbeing (i.e. happiness or mood) of the patient and may, for example, be a wellbeing score. The measure of wellbeing may be normalized. As an example, the measure of wellbeing may be calculated as a score between 0 and 100, where 0 is indicative of the patient having a lowest feeling of wellbeing and 100 is indicative of the patient having a highest feeling of wellbeing. A new measure of wellbeing may be calculated periodically. For example, a measure of wellbeing may be calculated according to a predetermined schedule (e.g. daily), in response to receipt of new wellbeing data being input by the user 350, in response to receipt of a different user input at the user input interface 114, or as part of a method such as a method discussed in relation to FIG. 5 and FIG. 6. The measure of wellbeing may therefore change over time, for example increasing as the treatment regime progresses.

The measure of wellbeing may be determined based on the most recent wellbeing data received by the mobile device 100, or it may be determined at least in part based on historical wellbeing data. As an example, a moving average of wellbeing data may be used to determine the measure of fitness. In some cases the moving average may be based on a predetermined number of the most recently received wellbeing data inputs (e.g. the last ten inputs of wellbeing data received by the mobile device 100). In some cases the moving average may be based on an average over a predetermined time period, for example the average wellbeing over the last seven days. The wellbeing data may be weighted such that more recent wellbeing data is given a greater weighting than older wellbeing data.

Each time a measure of wellbeing has been calculated, it can be stored in the memory 104 of the device 100 and/or transmitted to the server arrangement 240 for storage. The measure of wellbeing may be stored in association with a timestamp representing a time and/or date at which the measure of wellbeing was determined, or a time and/or date that wellbeing data used to calculate the measure of wellbeing was input by the user 350. The stored measurements of wellbeing and the associated timestamp information may provide a record of the patient's changing wellbeing. The record may be used to assess how the patient's wellbeing has changed over time, for example whether it is improving or worsening as the treatment regime progresses. The record of measurements of wellbeing may be output to the user as a visualization, as discussed later, or transmitted to the server arrangement 240 for analysis.

The side effect data comprises data indicative of any side effects experienced by the patient, wherein the side effects are associated with the medicament they are being administered during the treatment regime. Example side effects may include headaches, drowsiness, lethargy and disturbed vision, but the side effects are not limited to such examples and other side effects may be considered.

The device 100 may provide a graphical user interface (GUI) on the display 112 that assists the user 350 in providing the side effect data via the user input interface 114. For example, the GUI may request specific side effect data from the user 350 and may provide means for the user to input the requested data. For example, the GUI may comprise a prompt asking the user 350 if they have experienced any migraines in the past seven days. The user may input data responsive to the prompt via the user input interface 114. For example, the user may use a keypad to select a ‘yes’ or ‘no’ option displayed on the display 112, or if the display is a touchscreen display then the user may select a user-selectable GUI element provided on the display 112 by the device 100. The device 100 may request closed answers from the user 350 (i.e. ‘yes’/‘no’ answers) or open answers (i.e. a numerical value).

The device 100 may data to the server arrangement 240. The data may comprise any of the aforementioned information and data received by the device such as the physiological data, dose information, weight data, wellbeing data, side effect data, or any other data derived therefrom, such as the measure of fitness (e.g. fitness score), measure of bodyweight (e.g. BMI) or wellbeing score. The data may be sent to the server arrangement 240 for processing and/or storing. The data may be added to a record for the patient so that the data may be monitored over time. The data may also be compared with corresponding data from other patients to modify or improve the treatment regimen. The acquisition and sharing of patient wellbeing and side effect data may further assist in studying and improving the treatment regimen.

Some of the data, or data derived therefrom, may also be transmitted from the mobile device 100 to a social media platform 250, in some examples via the server arrangement 240. The patient may choose whether to post (i.e. publish) some of the data on the social media platform 250. The posted data may be visible by other users of the social media platform 250. The other users of the social media platform 250 may be able to post their own information in response to the data posted by the user of the mobile device 100, for example comments, virtual ‘likes’ or messages of encouragement for the user. The user may be able to view the messages posted on the social media platform 250 by the other users, which may provide encouragement for the user to continue their treatment regime.

Some of the data collected and/or determined by the device 100 may be output for display to the user so that the user may monitor changes in their data over time.

The data may be output for display on the display 112 of the mobile device 100 or another display, such as the display of a different laptop or tablet.

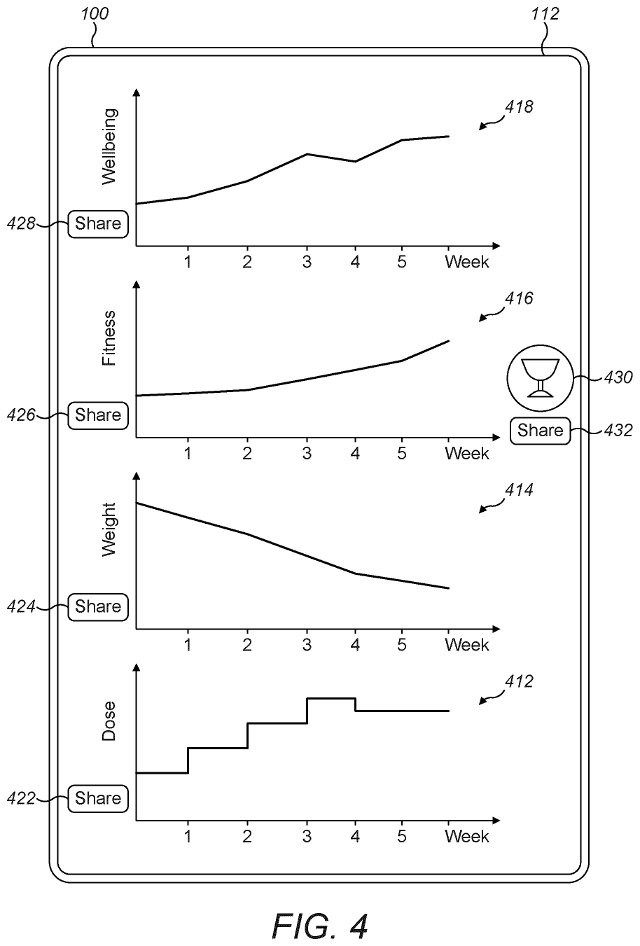

FIG. 4 shows patient data output to a user using the display 112 of the mobile device 100. The data is output in the form of one or more graphs 412, 414, 416, 418. One of the graphs 412 may illustrate the dose over time. For example, the graph 412 in FIG. 4 shows the dose increasing until week four, before decreasing, then remaining constant. This may be indicative of the dose being titrated to find an optimum dose for the patient, as discussed later. One of the graphs 414 may illustrate the measure of bodyweight over time. For example, the graph 414 in FIG. 4 shows the weight of the patient decreasing over time, as the treatment progresses. One of the graphs 416 may illustrate the measure of fitness over time. For example, the graph 416 in FIG. 4 shows the patient's fitness increasing over time, as the treatment progresses. One of the graphs 418 may illustrate the measure of wellbeing over time. For example, the graph 418 in FIG. 4 shows the patient's wellbeing increasing until week four, before decreasing slightly, then increasing again. It can be seen from graph 418 and graph 412 that the decrease in the patient's wellbeing in week four coincides with a decrease in the dose. This may be a result of the patient's wellbeing being taken into account when determining a new, adjusted dose of medicament to administer, as discussed elsewhere in this disclosure. The patient's wellbeing has decreased and as a result the dose has been adjusted to decrease slightly compared to the previously administered dose. The patient's wellbeing can then be seen to increase the following week, after the dose has been decreased.

While FIG. 4 shows four distinct graphs 412, 414, 416, 418, in other examples more than one data set can be illustrated in the same graph. Other forms of visualization may also be used to present the data. The patient may be encouraged to adhere to the treatment regimen in response to viewing the visualizations of the data, for example as provided by the graphs 412, 414, 416, 418. The patient may be encouraged by seeing the measure of fitness generally increasing over time, the measure of bodyweight generally decreasing over time, and/or the wellbeing score generally increasing over time.

The device 100 may provide a graphical user interface (GUI) element 422, 424, 426, 428 on the display 112, the selection of which by a user causes the device 100 to share the associated data with the server arrangement 240 and/or social media platform 250. For example, a user may interact with the user input interface 114 to select GUI element 426, thereby causing the device 100 to share the fitness data, for example by posting the fitness data to the social media platform 250. Likewise, selection of a GUI element 422 may share dose data, selection of a GUI element 424 may share weight data, and selection of a GUI element 428 may share wellbeing data. In some examples, selection of one GUI element may share more than one type of data.

In some examples the user may receive a virtual award 430 on their mobile device 100 based on the data collected or determined by the system 200. The virtual award 430 may be visible on the display 112 of the device 100 as a GUI element and may provide encouragement to the user to continue with the treatment regime. The patient may earn the award 430 when a predefined target is achieved, for example when the measure of fitness or measure of bodyweight meet a predetermined criteria, such as falling above or below a threshold. The user may receive a virtual reward 430 based on a trend of the of the measure of fitness or measure of bodyweight, for example if the measure of fitness has increased over two or more consecutive determinations or the measure of bodyweight has decreased over two or more determinations. In the example of FIG. 4, the user has received a virtual award 430 in relation to their measure of fitness. This is indicated by the award 430 being displayed in association with the fitness graph 426. The award 430 may have been received in response to the user's measure of fitness exceeding a predetermined threshold.

The virtual award 430 may be posted by the user to the social media platform 250. For example, the device 100 may provide a notification on the display 112 inviting the user to post the award 430. The award 430 may be posted in response to user selection of a graphical user interface (GUI) element 432 on the display 112. Other users of the social media platform 250 may be able to comment on or otherwise interact with the award 430, thereby providing encouragement for the patient.

FIGS. 5 and 6 are flow charts illustrating operation of the monitoring application 110 when the machine readable instructions are executed by the processing arrangement 102 of the mobile device 100. The flow charts indicate how the monitoring application 110 and the mobile device 100 interact and operate to provide a monitoring device. The steps are performed by the processing arrangement 102 of the mobile device 100 under control of the monitoring application 110 stored in the memory 104.

The dose of medicament administered to a patient may need to be adjusted over time in order to find an optimal dose for the patient. This is known as titration. FIG. 5 illustrates a titration method to be performed by the mobile device 100 in accordance with aspects of the disclosure. The titration method allows the patient to determine a new, adjusted dose of medicament to be administered based on a previously administered dose, wellbeing data, side effect data, and statistical data such as the measure of fitness and measure of bodyweight.

The titration is started in step 510. Step 510 may be performed in response to a user input provided at the user input interface 114, for example a user selection of a graphical user interface (GUI) element provided on the display 112. In some examples, the titration may be started without a user input. For example, the titration method may be started according to a schedule, such as at 10 AM daily. In some examples the titration is performed each time a dose of medicament is to be administered, while in other examples the titration may be performed during a ‘set-up’ period, for example during the first few weeks of a patient starting the treatment regimen.

In step 520, the device receives dose information as discussed previously. The dose information may indicate a previous dose administered to the patient during the treatment regimen, for example administered using the injection device 210. The dose information may comprise a dose value and in some examples a time and/or date that the dose was injected. The dose information may be received from the injection device 210 or it may be entered by the user, for example through the user input interface 114. In some examples, the dose information may have already been stored in the device 100 such as in the memory 104 of the device 100, and the device 100 retrieves the stored dose information from the memory 104. In other examples, the device 100 can receive the dose information from another source such as the server arrangement 240. The device 100 may send a request to the injection device 210 and/or server arrangement 240 that causes the injection device 210 and/or server arrangement 240 to transmit the dose information to the device 100.

In step 530, the device 100 receives wellbeing data associated with the patient. The wellbeing data may be input by the user in response to a prompt by the device as discussed previously, or it may be retrieved from a memory 104 of the device or the server arrangement 240.

In step 540 the device 100 receives side effect data. The side effect data may be input by the user in response to a prompt by the device as discussed previously, or it may be retrieved from a memory 104 of the device or the server arrangement 240.

In step 550 the device receives statistical data comprising at least one of the measure of fitness and the measure of bodyweight.

If the device receives a measure of fitness then this may involve the mobile device 100 requesting physiological data from the fitness tracking device 220 and determining a measure of fitness as discussed previously. In other examples, the mobile device 100 may not need to request the physiological data from the fitness tracking device 220 and may determine the measure of fitness based on physiological data already stored in the device 100. In other examples, the mobile device 100 may retrieve a measure of fitness that has already been determined and stored in the device 100, for example the most recently measure of fitness.

If the device receives a measure of bodyweight then this may involve the mobile device 100 requesting patient weight data from the balance 230 and determining a measure of bodyweight as discussed previously. In other examples, the mobile device 100 may not need to request the bodyweight data from the balance 230 and may determine the measure of bodyweight based on weight data already stored in the device 100. In other examples, the mobile device 100 may retrieve a measure of bodyweight that has already been determined and stored in the device 100, for example the most recently measure of bodyweight.

In step 560 the device determines an adjusted dose to be administered to the patient. This is the next dose of medicament that the patient should receive from the injection device 210. The adjusted dose is determined based at least in part on the dose information, wellbeing data, and the statistical data. The adjusted dose is determined using an algorithm that takes into account each of the dose information, wellbeing data, the side effect data and the statistical data. The algorithm may determine the adjusted dose by adjusting the previous dose indicated by the dose information based on the wellbeing data, the side effect data and the statistical data.

A measure of wellbeing is determined based on the wellbeing data as discussed previously, and it is the measure of wellbeing that is used to determine the adjusted dose. The previous dose may be increased, decreased or maintained to produce the adjusted dose, based on the measure of wellbeing. For example, if the measure of wellbeing is above a predetermined threshold then the algorithm may determine the adjusted dose by increasing the previous dose (or decreasing the dose, if appropriate for the drug concerned). However, if the measure of wellbeing is below a predetermined threshold then the algorithm may determine the adjusted dose by decreasing the previous dose (or increasing the dose, if appropriate for the drug concerned). If the measure of wellbeing is within a range of predetermined values then the algorithm may not increase or decrease the previous dose to determine the adjusted dose.

A measure of fitness is determined based on the physiological data as discussed previously, and it is the measure of fitness that is used to determine the adjusted dose. The previous dose may be increased, decreased or maintained to produce the adjusted dose, based on the measure of fitness. For example, if the measure of fitness is above a predetermined threshold then the algorithm may determine the adjusted dose by increasing the previous dose (or decreasing the dose, if appropriate for the drug concerned). However, if the measure of fitness is below a predetermined threshold then the algorithm may determine the adjusted dose by decreasing the previous dose (or increasing the dose, if appropriate for the drug concerned). If the measure of fitness is within a range of predetermined values then the algorithm may not increase or decrease the previous dose to determine the adjusted dose.

The algorithm may also determine the adjusted dose based on the side effect data, for example increasing, decreasing or maintaining the previous dose to obtain the adjusted dose based on the side effect data. Where the side effect data indicates that no side effects, or in some cases few very side effects or relatively minor side effects, have been experienced by the patient then the previous dose may be increased (or decreased, if appropriate for the drug concerned) to provide the adjusted dose, or in some cases the previous dose may be maintained (i.e. not adjusted). Where the side effect data indicates that side effects have been experienced by the patient, for example where the side effects are not minor in nature, then the previous dose may be decreased (or increased, if appropriate for the drug concerned) to provide the adjusted dose. The size of the adjustments made to the previous dose to determine the adjusted dose may be weighted depending on the type of the side effect. For example, serious side effects such as occurrences of vomiting may be more greatly weighted such that they have a larger effect on the adjusted dose determination than less serious side effects such as minor headaches. The size of the adjustments made to the previous dose to determine the adjusted dose may be weighted depending on the frequency of occurrence of the side effect and/or the intensity of the side effect. For example, if the patient provides side effect data indicative of frequent headaches then this may be more greatly weighted such that it has a larger effect on the adjusted dose determination than if the headaches were less frequent. Similarly, side effect data indicative of intense headaches may be more greatly weighted than side effect data indicative of minor headaches.

In step 570 the determined dose is output. This may comprise the device 100 causing the determined adjusted dose value to be displayed on the display 112 of the device 100, such as in units of the drug concerned. The patient may then view the output adjusted dose value and dial that dose into the injection device 210 ready for the next injection. In some examples, outputting the determined dose may comprise the device 100 transmitting a signal indicative of the determined dose to the injection device 210, which signal may cause the injection device 210 to display the determined dose on a display of the injection device 210 and/or cause the injection device 210 to be automatically dialled for the adjusted dose value. The patient may then inject the determined dose of medicament.

FIG. 6 illustrates a method to be performed using the mobile device 100 according to aspects of the present disclosure.

In optional step 610, the device 100 causes an alert to be output for indicating to the user that an injection is due. The alert may comprise an audio alert for example using a speaker of the device 100, a visual alert for example using the display 112 of the device 100, and/or a haptic alert for example using a haptic transducer of the device 100. In some examples the device 100 causes the alert to be output using another device such as the injection device 210, fitness tracking device 220 or balance 230. This may be in addition to or instead of outputting an alert from the device 100 itself. The device 100 may cause the alert to be output according to a predetermined schedule, for example daily, periodically every predetermined number of hours, or at a predetermined time and/or date.

In step 612, the device 100 requests dose information and in step 614 the device receives the dose information, for example as discussed previously. In some examples the device 100 does not need to request the dose information and so step 612 is not performed. As discussed previously in relation to step 520 of FIG. 5, the dose information may indicate a previous dose administered to the patient, for example using the injection device 210. The dose information may comprise a dose value and in some examples a time and/or date that the dose was injected.

The dose information may be received in step 614 from the injection device 210, in response to the device 100 transmitting a request for the information to the injection device 210 in step 612. However in some cases no request is transmitted to the injection device 210, and instead the injection device 210 transmits the dose information automatically, for example in response to an injection being performed.

In some examples step 614 comprises receiving the dose information from a user, for example via the user input interface 114. In this case, step 612 may comprise causing a prompt to be displayed on the display 112 of the device 100 requesting the dose information from the user. The user may input the dose information via the user input interface 114 in response to the prompt or otherwise.

In some examples, the dose information may have already been stored in the device 100 such as in the memory 104 of the device 100, and therefore in step 614 the device 100 retrieves the stored dose information from the memory 104. Step 612 may therefore not be required.

In other examples, the device 100 can receive the dose information from another source such as the server arrangement 240. The device 100 may send a request in step 612 to the server arrangement 240 that causes the server arrangement 240 to transmit the dose information to the device 100, where it is received in step 614.

In step 6166, the device 100 requests physiological data from the fitness tracking device 220, as discussed previously. In step 618, the device 100 receives the physiological data from the fitness tracking device 220. The physiological data may comprise at least one of a heart rate, a blood pressure and a step count of the user. In some examples step 618 is performed without step 616. In this case, the fitness tracking device 220 may automatically transmit the physiological data to the mobile device 100.

In step 620, the device 100 requests weight data from the balance 230, as discussed previously. In step 622, the device 100 receives the weight data from the balance 230. In some examples step 622 is performed without step 620. In this case, the balance 230 may automatically transmit the weight data to the mobile device 100.

In step 624, the device requests wellbeing data, as discussed previously. Requesting the wellbeing data may comprise providing a graphical user interface element on the display of the device 100 which indicates to the user that wellbeing data is required. However, in some cases step 624 is not performed. In step 626, the device receives the wellbeing data. The wellbeing data may be received via user input into the user input interface 114 of the mobile device 100.

In step 628, the device requests side effect data, as discussed previously. Requesting the side effect data may comprise providing a graphical user interface element on the display of the device 100 which indicates to the user that side effect data is required. However, in some cases step 628 is not performed. In step 630, the device receives the side effect data. The side effect data may be received via user input into the user input interface 114 of the mobile device 100.

In step 632, the device determines a measure of fitness such as a fitness score based on the received physiological data, for example as discussed previously.

In step 634, the device determines a measure of patient bodyweight such as a BMI based on the received weight data, as discussed previously. In some examples the measure of bodyweight may simply be the mass of the patient, for example in kilograms or pounds.

In step 636, the device determines a measure of wellbeing based on the received wellbeing data, as discussed previously. The measure of wellbeing is a quantitative indication of the wellbeing of the patient, for example a wellbeing score. The measure of wellbeing may be based on the most recent wellbeing data, or it may be based on the most recent wellbeing data in addition to historical wellbeing data, for example by calculating a moving average of the wellbeing data, as discussed previously.

In step 638, the device 100 may store any of the aforementioned data, information, or statistics derived therefrom. The data may be stored in the memory 104 of the device and/or transmitted by the device to the server arrangement 240 for storage at the server arrangement 240, in the cloud, or elsewhere. The data may be stored with associated time and/or date information. The data may form part of a record for that particular patient so that changes in the data may be monitored over time.

In optional step 640, the device 100 determines a new, adjusted dose of medicament for the patient to inject, for example as previously described in relation to step 560 of FIG. 5. Step 640 may comprise any of the steps of FIG. 5 required to determine the adjusted dose. Some of these steps may have already been performed during the method of FIG. 6 and so do not need to be repeated in the method of FIG. 6. In some examples, the patient may have already injected a dose of medicament using the injection device before step 612 and so step 640 is not required.

In step 642, the device 100 outputs data. The data output by the device 100 may comprise one or more of the measure of wellbeing, the measure of bodyweight, the measure of fitness, the previous dose indicated in the dose information, the new determined dose, and any associated time/date data. The data may be output to the display 112 in the form of a visualization of the data, such as a graph as described previously in relation to FIG. 4. Alternatively or additionally, the data may be output to a server arrangement 240 as discussed previously, and/or a social media platform 250 as discussed previously.

While the above steps have been discussed in a particular order, it should be noted that the disclosure is not limited to that particular order and that various steps may be performed in a different order, or performed simultaneously to other steps. Similarly, some steps may be omitted or additional steps performed. For example, the various data may be requested and/or received in a different order to that discussed above and shown in FIG. 5 and FIG. 6.

The device 100 may also allow for the tracking of food intake by a user. The device 100 may provide a graphical user interface on the display 112 allowing the user to input food intake information. The graphical user interface may be provided in response to a user selecting a graphical user interface element on the display 112 of the device 100 using the user input interface 114, or may be provided as part of the method of FIG. 5 or FIG. 6. The food intake information may comprise an indication of a foodstuff consumed by the user and/or nutritional data associated with said foodstuff such as carbohydrate content in grams. The food intake information may be stored by the device 100 along with timestamp information indicating the time and/or date the foodstuff was consumed. The timestamp information may be input by the user through the user input interface 114 or may be determined by the mobile device 100. The food intake information may be stored in the memory 104 of the device 100 and/or transmitted to the server arrangement 240 for storage and/or processing. The food intake of the patient may therefore be tracked over time. The mobile device 100 or server arrangement 240 may determine food intake recommendations for the patient based on the historic food intake information. These recommendations may be displayed on the mobile device 100 via the display 122.

Similarly, the mobile device 100 and/or server arrangement 240 may determine fitness recommendations based on the historical physiological data received by the mobile device 100 and display the recommendations on the display 112 of the mobile device 100. For example, the mobile device 100 or server arrangement 240 may determine based on historic physiological data (or measures of fitness) that the patient's fitness is decreasing and may therefore provide a recommendation to the user such as requesting that the user try to increase their step count. The mobile device 100 may also output encouragement to the user based on the measure of fitness or measure of bodyweight, for example by providing a message on the display 112 encouraging the user to ‘keep going’ with their treatment regime.

If the wellbeing data and determined dose data is output to the server arrangement 240 then this data may be used by a health care professional to improve the treatment regime, for example by improving the algorithm.

The present disclosure is described with reference to diabetes and chronic weight management, but this is not intended to be limiting and the teaching herein may equally well be deployed with respect to other diseases or health conditions.

The present disclosure is described in the context of a computer program implemented in a mobile device 100, but this is not intended to be limiting and the computer program may equally well be implemented in another suitable apparatus. For instance, the apparatus may equally well be implemented in another mobile device 100, such as a PDA, a tablet computer of any kind, or a medical device, such as a blood glucose meter device. Alternatively, the computer program may be implemented in another suitable apparatus, such as a PC.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present disclosure, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

1.-15. (canceled)

16. A computing device comprising a memory and a processing arrangement, the memory storing instructions which, when executed by the processing arrangement, cause the device to:
receive dose information comprising a previous dose ejected by an injection device;
determine a measure of wellbeing for a patient based on received wellbeing data;
determine at least one of:
a measure of fitness for the patient based on physiological data received from fitness tracking device; or
a measure of bodyweight for the patient based on weight data received from a balance;
determine an adjusted medicament dose based at least in part on the dose information, the measure of wellbeing, and the at least one of the measure of fitness or the measure of bodyweight; and
output the adjusted medicament dose.

17. The device according to claim 16, wherein the instructions, when executed by the processing arrangement, further cause the device to output at least the wellbeing data and the dose information comprising the previous dose to a server arrangement for storage.

18. The device according to claim 16, wherein the physiological data comprises pulse data and wherein the instructions, when executed by the processing arrangement, cause the device to determine the measure of fitness based on the pulse data.

19. The device according to claim 16, wherein the physiological data comprises blood pressure data and wherein the instructions, when executed by the processing arrangement, cause the device to determine the measure of fitness based on the blood pressure data.

20. The device according to claim 16, wherein the physiological data comprises step count data and wherein the instructions, when executed by the processing arrangement, cause the device to determine the measure of fitness based on the step count data.

21. The device according to claim 16, wherein the instructions, when executed by the processing arrangement, further cause the device to:
output at least one of the measure of wellbeing, the measure of fitness, or the measure of bodyweight for display.

22. The device according to claim 21, wherein the at least one of the measure of wellbeing, the measure of fitness, or the measure of bodyweight for display are displayed along with one or more previous measures of wellbeing, measures of fitness, or measures of bodyweight.

23. The device according to claim 16, wherein the instructions, when executed by the processing arrangement, further cause the device to:
receive side effect data indicative of one or more side effects experienced by the patient; and
determine the adjusted medicament dose based at least in part on the side effect data.

24. The device according to claim 16, wherein the instructions, when executed by the processing arrangement, further cause the device to:
determine whether the measure of fitness meets a first predetermined threshold;
determine whether the measure of bodyweight meets a second predetermined threshold; and
in response to determining that the measure of fitness meets the first predetermined threshold or that the measure of bodyweight meets the second predetermined threshold, output a virtual award for display at the device.

25. The device according to claim 16, wherein the instructions, when executed by the processing arrangement, further cause the device to receive food intake information input by a user and store the received food intake information.

26. The device according to claim 16, wherein the instructions, when executed by the processing arrangement, further cause the device to publish data corresponding to at least one of the measure of wellbeing, the measure of fitness, or the measure of bodyweight to a social media platform.

27. A system comprising:
a computing device comprising a memory and a processing arrangement, the memory storing instructions which, when executed by the processing arrangement, cause the device to:
receive dose information comprising a previous dose ejected by an injection device;
determine a measure of wellbeing for a patient based on received wellbeing data;
determine at least one of:
a measure of fitness for the patient based on physiological data received from fitness tracking device; or
a measure of bodyweight for the patient based on weight data received from a balance;
determine an adjusted medicament dose based at least in part on the dose information, the measure of wellbeing, and the at least one of the measure of fitness or the measure of bodyweight; and
output the adjusted medicament dose;
a fitness tracking device configured to transmit physiological data to the device; and
a balance configured to transmit weight data to the device,
wherein the device is configured to determine an adjusted medicament dose based at least in part on the physiological data and the weight data received by the device.

28. The system according to claim 27, further comprising an injection device configured to transmit dose information to the device, wherein the device is configured to determine the adjusted medicament dose based at least in part on the dose information.

29. A computer-implemented method comprising:
receiving dose information comprising a previous dose ejected by an injection device;
determining a measure of wellbeing for a patient based on received wellbeing data;
determining at least one of:
a measure of fitness for the patient based on physiological data received from fitness tracking device; or
a measure of bodyweight for the patient based on weight data received from a balance;
determining an adjusted medicament dose based at least in part on the dose information, the measure of wellbeing, and the at least one of the measure of fitness or the measure of bodyweight; and
outputting the adjusted medicament dose.

30. The method of claim 29, further comprising: causing the device to output at least the wellbeing data and the dose information comprising the previous dose to a server arrangement for storage.

31. The method of claim 29, wherein the physiological data comprises pulse data and wherein the method further comprises causing the device to determine the measure of fitness based on the pulse data.

32. The method of claim 29, wherein the physiological data comprises blood pressure data and wherein the method further comprises causing the device to determine the measure of fitness based on the blood pressure data.

33. The method of claim 29, wherein the physiological data comprises step count data and wherein the method further comprises causing the device to determine the measure of fitness based on the step count data.

34. The method of claim 29, further comprises causing the device to output at least one of the measure of wellbeing, the measure of fitness, or the measure of bodyweight for display.

35. One or more computer-readable storage media storing instructions which, when executed by one or more computers, cause the one or more computers to perform the following operations:
receiving dose information comprising a previous dose ejected by an injection device;
determining a measure of wellbeing for a patient based on received wellbeing data;
determining at least one of:
a measure of fitness for the patient based on physiological data received from fitness tracking device; or
a measure of bodyweight for the patient based on weight data received from a balance;
determining an adjusted medicament dose based at least in part on the dose information, the measure of wellbeing, and the at least one of the measure of fitness or the measure of bodyweight; and
outputting the adjusted medicament dose.
